(19) United States
(12) Patent Application Publication
(10) Pub. No.: US 2007/0191876 A1
(43) Pub. Date: Aug. 16, 2007

(54) Upper Airway Device and Method

(75) Inventors: William R. Dubrul, Redwood City, CA (US); Richard Eustis Fulton III, Grand Junction, CO (US)

Correspondence Address: HAYNES BEFFEL & WOLFELD LLP, P O BOX 366, HALF MOON BAY, CA, 94019, US

(73) Assignee: Genesis Technologies LLC, Belmont, CA (US)

(21) Appl. No.: 11/692,082

(22) Filed: Mar. 27, 2007

Related U.S. Application Data
(62) Division of application No. 10/423,068, filed on Apr. 25, 2003.
(60) Provisional application No. 60/377,951, filed on May 2, 2002.
(60) Provisional application No. 60/435,125, filed on Dec. 18, 2002.

(51) Int. Cl. A61M 31/00; A61M 29/00; A62B 9/06

(52) U.S. Cl. 606/199; 128/207.14; 604/48

(57) ABSTRACT

A medical device includes a radially expandable and contractible tubular element, having proximal and distal ends, sized to pass through the nostril and into the user's upper airway when in the radially contracted condition. The support element may create an air passageway therethrough when in a radially expanded condition. The tubular element may comprise a porous filter section for filtering air passing through the air passageway. The tubular element may comprise a body material and an agent releasable from the body material for delivery to tissue of the user when the tubular element is radially expanded.

### CROSS-REFERENCE TO OTHER APPLICATIONS

This application is a divisional of U.S. patent application Ser. No. 10/423,068, filed 25 Apr. 2003, which claims the benefit of U.S. Provisional Patent Application No. 60/377,951 filed 2 May 2002 and U.S. Provisional Patent Application No. 60/435,125 filed 18 Dec. 2002. See also: U.S. Pat. No. 6,450,989; U.S. Pat. No. 6,258,115; U.S. patent application Ser. No. 10/051,848 filed Jan. 17, 2002 entitled “Particle removing medical device and method,” published on 31 Oct. 2002 as Publication No. US-2002-0161392-A1.

### FEDERALLY SPONSORED RESEARCH OR DEVELOPMENT

None.

### BACKGROUND OF THE INVENTION

Obstructive sleep apnea is characterized by episodes of partial or complete nasopharyngeal obstruction during sleep. The term apnea is defined as the cessation of air flow for a minimum of 10 seconds. These periods of apnea are frequently associated with sleep fragmentation and a drop in oxygen saturation. The term hypopnea is defined as a 30 to 50% reduction in airflow for a minimum of 10 seconds. Both apnea and hypopnea cause respiratory effort-related arousals and disordered sleep. When these apneras and hpopneas are combined with symptoms such as daytime somnolence, the terms obstructive sleep apnea-hypopnea syndrome or obstructive sleep apnea are utilized. These episodes of apnea and hypopnea lead to daytime sleepiness and other pathological manifestations including stroke, cardiac arrhythmias, hypertension, sudden death, psychological syndromes, depression, hyperactivity, and others.

The upper airway resistance syndrome is a breathing disorder during sleep in which there is increased breathing effort during periods of increased upper airway resistance not resulting in apneas or hypopnic episodes. These patients however experience daytime somnolence as well.

Epidemiologically, it has been estimated that 5% of the adult population under 60 years old meet the criteria of obstructive sleep apnea syndrome. It is even more common in the elderly, and with the “graying of America,” the incidence is certainly going to increase. Snoring is a sleep related breathing disorder estimated to affect 10-86% of the population and can be related to OSA syndrome. More commonly, it causes nighttime awakenings and daytime somnolence in the sleep partner. Increased incidence of automobile accidents has been found in sleep partners of snorers, as well as diminished job productivity. It has been estimated that up to 30 million individuals in the U.S. suffer from some form of OSA syndrome, upper airway resistance syndrome, problematic snoring, and other sleep related breathing disorders.

Treatment of these disorders has been varied, mainly due to either the lack of success and the lack of compliance. An effective treatment is continuous positive airway pressure (“CPAP”), which is administered by means of nasal mask. Room air is pressurized by a mechanical device and forced into the nasal passageway. This distends the tissues in the upper airway and overcomes the obstruction, providing for patency. While advancements have been made to improve the comfort and sealing of the device, poor acceptance and compliance have limited its utility.

Surgical techniques have been utilized, but are invasive and are effective in a minority of patients. Oral appliances and retention devices have also been utilized, but with very limited success. Insertion of nasophanyngeal tubes has resulted in effective treatment of the OSA episodes and diminished daytime somnolence. They are impractical as they are rigid, uncomfortable, and need to be inserted by a medical professional, not the patient. Zammit, in U.S. Pat. No. 6,328,753, describes a novel nasopharyngeal tube which is collapsible for insertion, and subsequently expanded. While an improvement, it too has limitations, as it eliminates the moistening effect on inhaled air normally provided by the nasal mucosa. The contact of the plastic with large areas of mucosa may cause irritation, inflammation, and further mucosal edema limiting long-term compliance. It also suffers from the lack of a small delivery device.

### SUMMARY OF THE INVENTION

The present invention relates to medical devices and their methods of use. More specifically, one aspect of the present invention relates to devices which are particularly useful for repairing and/or serving as a conduit for body passageways requiring reinforcement, dilatation, disease prevention or the like. The present invention may provide temporary patency of the upper airway for breathing disorders, sleep apnea, and other obstructions of the nasoppharyneal region. Another aspect of the invention relates to the delivery of a therapy, that therapy being from a family of devices, drugs, or any of a variety of other elements, to a specific location within the body. More specifically, the therapy may be directed to systemic uptake through the nasal mucosa or along a path extending from the nasal cavity to the lungs. A further aspect of the invention may be used as a nasal filter to remove pollen, or other unwanted particulate matter, and non-particulate substances, such as noxious or unhealthy chemical compounds, bacteria and viruses, from the inhaled air.

A first aspect of the invention is directed to a device for maintaining the patency of a user's upper airway. The device includes a radially expandable and contractible tubular support element, having proximal and distal ends and means for selectively placing the tubular support element in a radially contracted condition and a radially expanded condition. The support element is sized to pass through the nostril and into the user's upper airway for positioning within the upper airway when in the radially contracted condition. The support element creates an air passageway through the support element between the proximal and distal ends when in the radially expanded condition.

A second aspect of the invention is directed to a method for maintaining the patency of a user's upper airway. A radially expandable and contractible tubular support element, having proximal and distal ends, is placed through a nostril and into the user's upper airway. The support element is positioned within the user's upper airway. The support element is radially expanded thereby creating an air passageway within the support element between the proximal and distal ends. The support element is radially contracted and the radially contracted support element is removed through the nostril.

A third aspect of the invention is directed to a device for filtering air passing through a user's upper airway. The device includes a radially expandable and contractible tubular element having proximal and distal ends. The tubular element is sized to pass through a nostril and into the user's upper airway to be positioned at a desired location within the user's upper airway so that when the tubular element is radially expanded, an air passageway is created within the support element between the proximal and distal ends. The tubular element comprises a porous filter section extending at least partially across the air passageway so to filter air passing through the air passageway.

A fourth aspect of the invention is directed to a method for filtering air passing through a user's upper airway. A radially expandable and contractible tubular element having proximal and distal ends is selected. The tubular element through a nostril and into the user's upper airway and is positioned at a desired location within the user's upper airway. The tubular element is radially expanded thereby creating an air passageway within the tubular element between the proximal and distal ends. Air passing through the air passageway is filtered.

A fifth aspect of the invention is directed to device for delivering an agent to tissue of a user. The device includes a radially expandable tubular element having proximal and distal ends. The tubular element is sized to pass through a nostril and into the user's upper airway to be positioned at a desired location within the user's upper airway so that when the tubular element is radially expanded, an air passageway is created within the support element between the proximal and distal ends. The tubular element comprises a body material and an agent releasable from the body material when the tubular element is radially expanded.

A sixth aspect of the invention is directed to a method for delivering an agent to tissue of a user. A radially expandable tubular element having proximal and distal ends is selected. The tubular element is placed through a nostril and into the user's upper airway. The tubular element is positioned at a desired location within the user's upper airway. The tubular element is radially expanded thereby creating an air passageway within the tubular element between the proximal and distal ends. The tubular element is chosen to include a body material and an agent releasable from the body material. The agent is released for delivery to tissue of the user.

One of the advantages of the invention is that it is simple in construction and can be made at a reasonable cost. This is important because even if the invention performs a function in some improved manner, it will not be widely used if it is considerably more costly than the alternatives available. Another advantage is that it is simple to use and in a very real sense simple to understand. This will encourage its adoption and use by patients and medical personnel. It will also tend to keep cost low. A further advantage is that the procedure for use is simple so that the device and procedure may be self administered by the patient.

Other features and advantages of the invention will appear from the following description in which the preferred embodiments have been set forth in detail in conjunction with the accompanying drawings.

### BRIEF DESCRIPTION OF THE DRAWINGS

FIG. 1 is an overall view of an upper airway device including an insertion sheath housing the tubular support element.

FIG. 2 illustrates the device of FIG. 1 with the insertion sheath partially removed allowing the distal end of the tubular support element to move from its radially contracted, restrained state to its relaxed, radially expanded state.

FIG. 3 illustrates the device of FIG. 1 after having been inserted through the user's nostril into the upper airway with the distal end positioned between the uvula and the posterior pharyngeal wall with the insertion sheath maintaining the tubular support element in a contracted condition.

FIG. 4 illustrates the device of FIG. 3 after the insertion sheath has begun to be removed thus permitting the tubular support element to move to its relaxed, radially expanded state thus anchoring the tubular support element in place.

FIG. 5 illustrates the result of having completely remove the insertion sheath from the tubular support element so that the tubular support element defines an air passageway therethrough.

FIG. 6 illustrates a second embodiment of the upper airway device of FIG. 1 in which the tubular element is placed in tension and thus in a reduced diameter state by the insertion of an elongate placement element through the interior of the tubular element.

FIG. 7 illustrates the tubular element of FIG. 6 after the elongate placement element has been removed permitting the tubular element to assume its relaxed, expanded diameter state.

FIG. 8 illustrates a third embodiment of the invention similar to that of FIG. 6 but in which the tubular element is shown in a relaxed, radially contracted state.

FIG. 9 illustrates the device of FIG. 8 in a radially expanded state caused by pulling on string-like placement elements secured to the distal end of the tubular element passing through the interior of the tubular element and passing out through the open proximal end of the tubular element.

FIG. 10 illustrates a fourth embodiment of the invention in which the tubular element is a Malecott-type tubular element in a relaxed, radially contracted state.

FIG. 11 illustrates the device of FIG. 10 placed in a radially expanded state using string-like placement elements and also illustrating filter material at the distal end of the tubular element.

FIG. 12 shows a fifth embodiment of the invention similar to FIG. 10 in which the tubular element is maintained in the radially contracted state through the use of an elongate placement element similar to that used with the FIG. 6 embodiment.

FIG. 13 illustrates the tubular element of FIG. 12 after the elongate placement has been remove allowing it to assume its relaxed, radially expanded state.

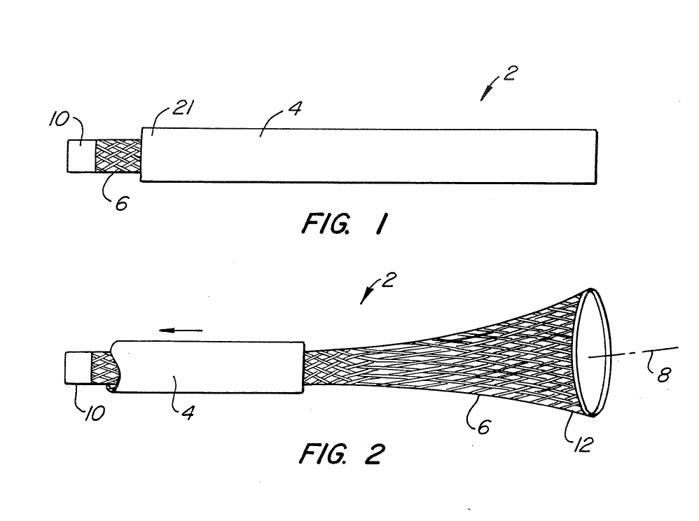

### DESCRIPTION OF THE SPECIFIC EMBODIMENTS

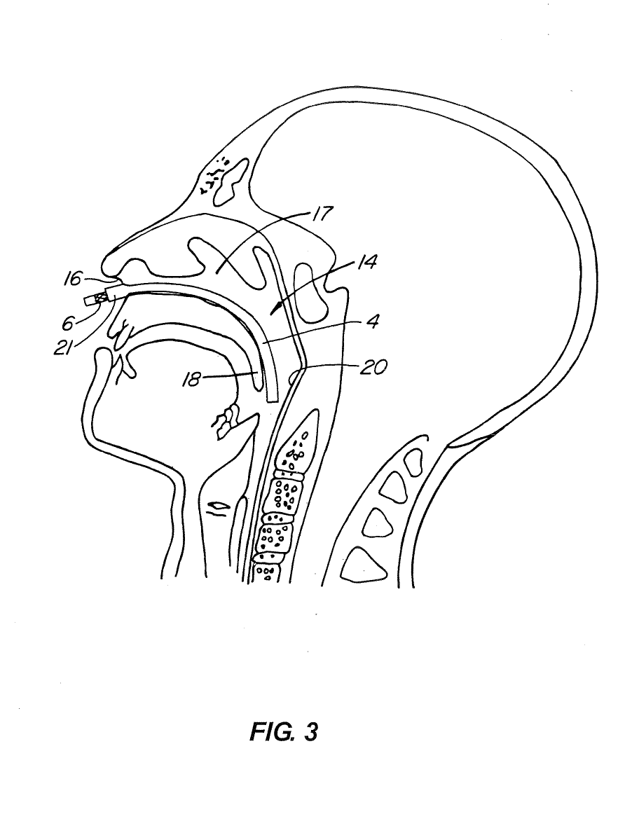

FIG. 1 illustrates an upper airway device 2 including a flexible insertion sheath 4 housing a porous tubular support element 6. Sheath 4 is preferably lubricated to facilitate insertion as discussed below. Tubular support element 6 defines an air passageway 8 from its proximal end 10 to its distal end 12. Tubular support element 6 is a radially expandable and contractible tubular mesh element similar to that shown in U.S. Pat. No. 6,221,006. The porous nature of support element 6 allows the air passing through it to be moistened by the mucosa of the nasal passageway. Distal end 10 is shown in this embodiment to be made of a nonexpandable material to aid in the grasping and manipulation of tubular support element 6 as will be discussed below with reference to FIGS. 3-5.

Tubular support element 6 is preferably formed as a mesh of individual non-elastic filaments (called “yarns” in the braiding industry). However, it can have some elastic filaments interwoven to create certain characteristics. The non-elastic yarns can be materials such as polyester, PET, polypropylene, polyamide fiber (Kevlar, DuPont), composite filament wound polymer, extruded polymer tubing (such as Nylon II or Ultem, commercially available from General Electric), stainless steel, Nickel Titanium (Nitinol), or the like so that axial shortening causes radial expansion of the braid. These materials have sufficient strength so that support element 6 will retain its expanded condition in the lumen of the body while positioned to maintain the airway. Further, all expandable mechanisms described heretofore can be manufactured using shape memory materials so that they are self expanding or even expandable when certain temperatures or thermal energies are delivered to the mechanisms. Such material characteristics can be accomplished with different programming methods such as, but not limited to Two Way Shape Memory (TWSM) alloys.

Support element 6 may be of conventional construction, comprising round filaments, flat or ribbon filaments, square filaments, or the like. Non-round filaments may be advantageous to decrease the axial force required for expansion to create a preferred surface area configuration or to decrease the wall thickness of the tubular braid. The filament width or diameter will typically be from about 0.5 to 50 mils, usually being from about 5 to 20 mils. Suitable braids are commercially available from a variety of commercial suppliers.

In a preferred embodiment, the filaments of support element 6 may be of a material that has physical properties that prevent the collapse of the nasopharyngeal tissues. They may contain a hydrophilic coating to prevent tissue damage and provide for comfort. Any one of a number of commercially available materials are available for this use.

FIG. 3 illustrates device 2 after having been placed along the user's upper airway 14 through nostril 16, through nasal cavity 17 and to a position between the uvula, at position 18, and the posterior pharyngeal wall, at position 20. The patient may do all of the above either blindly, or while standing in front of a mirror. A guide wire may be utilized to effect placement, as a soft guide wire will pass easily through the nostrils nasophanynx and into the oropharynx. The device may be passed over the guide wire or a monorail configuration may be used to facilitate the passage of the device. This maneuver will prevent the trauma of passing a 6-20 Fr. cannula through the nasal passages de novo. It will allow for a more comfortable placement and insure compliance. Support element 6 is now in place in a position that will prevent sleep apnea or other sleep related breathing disorders. Because the tubular structure formed by support element 6 is porous, the patient will be able to breath through both nostrils. Moreover, the nasal mucosa will be exposed to the flow of air into the lungs, moistening it. Moistening inhaled air is a primary purpose of the nasal mucosa, and upper airway device 2 promotes that important physiological action, as opposed to other nasophanyngeal tubes that isolate the air flow from the nasal mucosa because of the solid walls of those devices.

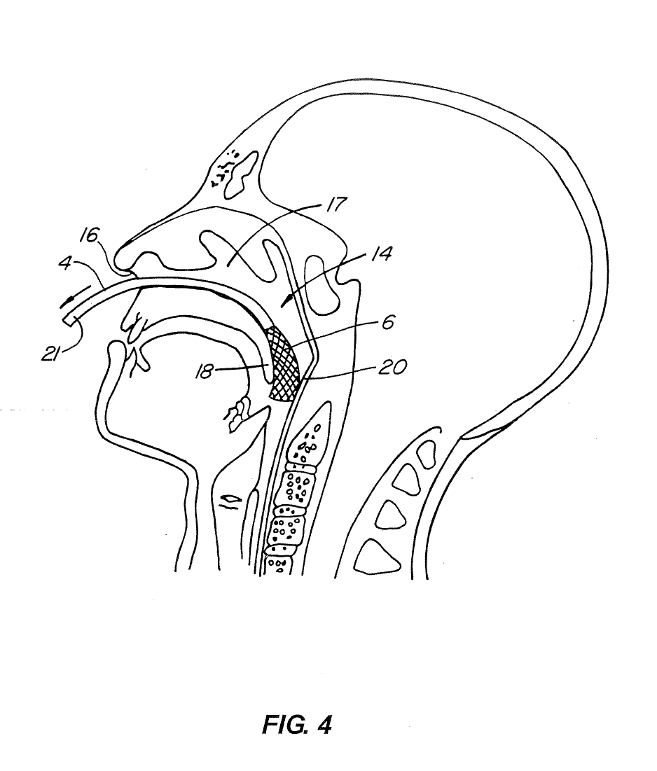

FIG. 4 illustrates the initial removal of insertion sheath 4 permitting outer end 12 of tubular support element 6 to naturally radially expand to help maintain support element 6 in position with outer end 12 between positions 18 and 20. During this removal process it may be necessary or desirable to grasp on proximal end 10 so that tubular support element 6 does not pull out of upper airway 14 along with insertion sheath 4.

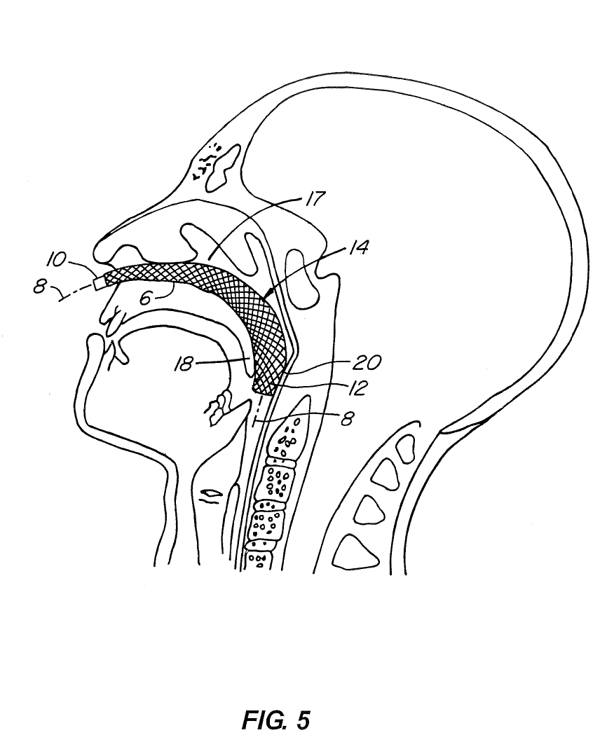

FIG. 5 illustrates tubular support element 6 fully radially expanded and defining air passageway 8 extending from proximal end 10 to distal end 12 of tubular support element 6. In this manner tubular support element 6 provides temporary patency of the upper airway 14.

To remove support element 6 from upper airway 14, the patient simply places sheath 4 over support element 6 and advances sheath 4 over support element 6. With traction on support element 6 and continued advancement of sheath 4, support element 6 will collapse into sheath 4 (be retracted into it) so that it is fully contained within. Device 2 is then removed from nostril 16. Insertion typically takes place before the user's sleep cycle while removal typically takes place after the user's sleep cycle.

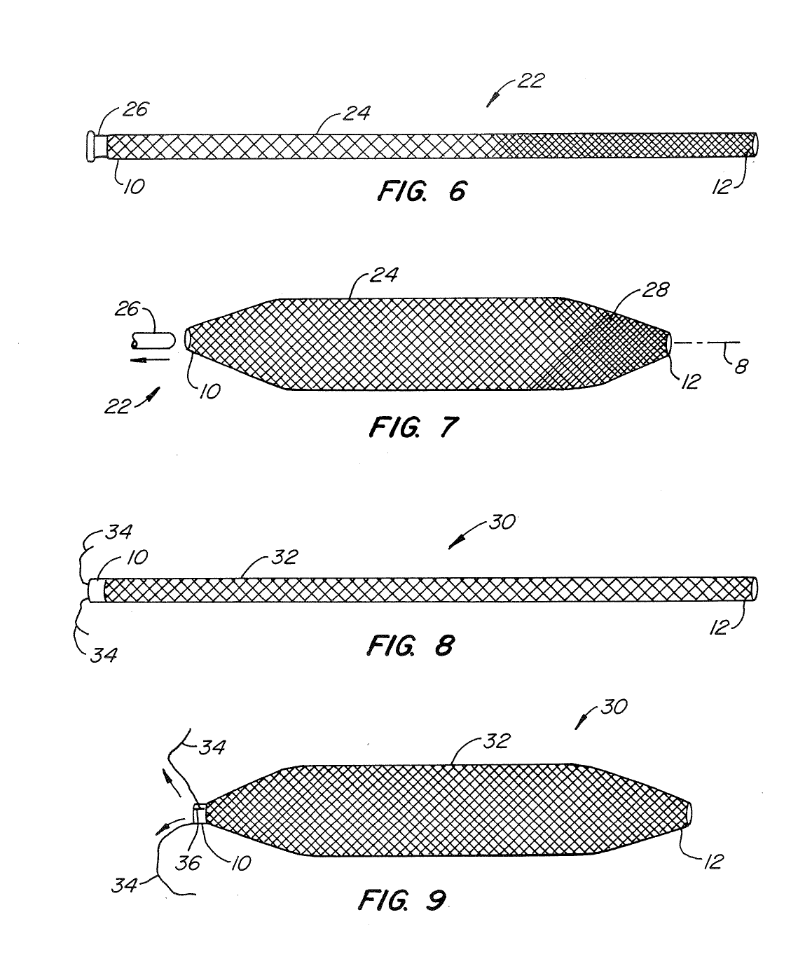

The remaining figures describe additional embodiments of the invention with like reference numerals referring to like elements. An upper airway device 22, including a tubular element 24, is shown in its relaxed, radially expanded condition in FIG. 7. Device 22 also includes a flexible elongate placement element 26 which passes through the open proximal end 10 of tubular element 24, extends through the interior of the tubular element and engages the generally closed distal end 12 of the tubular element. When this occurs the tubular element 24 placed in tension to resume its radially contracted condition of FIG. 6. In this radially contracted condition, device 22 may be passed through nostril 16, through nasal cavity 17 and positioned between uvula position 18 and posterior pharyngeal wall position 20 as in FIG. 3. When so positioned, elongate placement element 26 is removed from tubular element 24 to permit the tubular element to assume its relaxed, radially expanded condition of FIG. 7 thus providing an air passageway 8 therethrough.

As indicated in FIGS. 6 and 7, distal end 12 of tubular element 24 has a tighter weave than the remainder of the tubular element. This creates a porous filter section 28 at distal end 12 so that tubular element 24 filters air passing along air passageway 8. Porous filter section 28 may filter air simply by virtue of creating small openings to trap particles passing along air passageway 8. In addition, porous filter section 28, as well as the remainder of tubular element 24, can be created or treated so that it has an affinity for particles, such as pollen, or other substances, such as chemical compounds, to help remove them from the air passing along air passageway 8. Porous filter section 28 may be treated to have an electrostatic affinity for particles, a molecular attraction for molecules, a sticky or adhesive function or some other filtering mechanism. In the embodiment of FIG. 7, distal end 12, while porous, is substantially closed. Alternatively, tubular element 24 could be provided with a porous filter section 28 that extends only part way across the air passageway.

Tubular element 24 and/or porous filter section 28 may comprise an agent. The agent may include one or more of a for example, a drug therapy agent or a gene therapy agent. The agent may be of a type that is release directly to the tissue being contacted by tubular element 24. In addition, the agent may be of the type that is carried with the air passing along air passageway 8 for delivery to tissue at one or more locations located along a path extending from nasal cavity 17 to the user's lungs. The agent may also be of both types.

In some situations, especially when the purpose is to filter air and/or deliver an agent, it may not be necessary to have the tubular element extend from nostril 16 to positions 18, 20. For example, a tubular element similar to tubular element 24 of FIGS. 6 and 7 may include the entire porous filter section 28 but have a total length much shorter than tubular element 24 and the placeable entirely within nasal cavity 17.

FIGS. 8 and 9 illustrate an embodiment similar to the embodiment of FIGS. 6 and 7 but in which the tubular element assumes a relaxed, contracted state as shown in FIG. 8. Upper airway device 30 includes a tubular element 32 and a pair of string-like placement elements 34. Placement elements 34 extend from and are secured to distal end 12 of tubular element 32 pass through the interior of tubular element 32 and extend out though open proximal end 10. After appropriate placement along upper airway 14, string-like placement elements 34 are pulled thus causing tubular element 32 to assume the longitudinally contracted, radially expanded state of FIG. 9 thus creating air passageway 8 along upper airway 14. To maintain tubular element 32 in the radially expanded condition, string-like placement elements 34 are passed into slits 36 formed in proximal end 10 thus securing the placement elements to the proximal end. To remove tubular element 32, the user simply disengages the string-like placement elements 34 from slits 36 formed in proximal end 10 to permit tubular element 32 to resume its relaxed, radially contracted condition of FIG. 8 permitting easy removal. Tubular element 32 could be constructed so that it maintains a cylindrical shape when in its relaxed, radially contracted state, as in FIG. 8, as well as when in its contracted, radially expanded state.

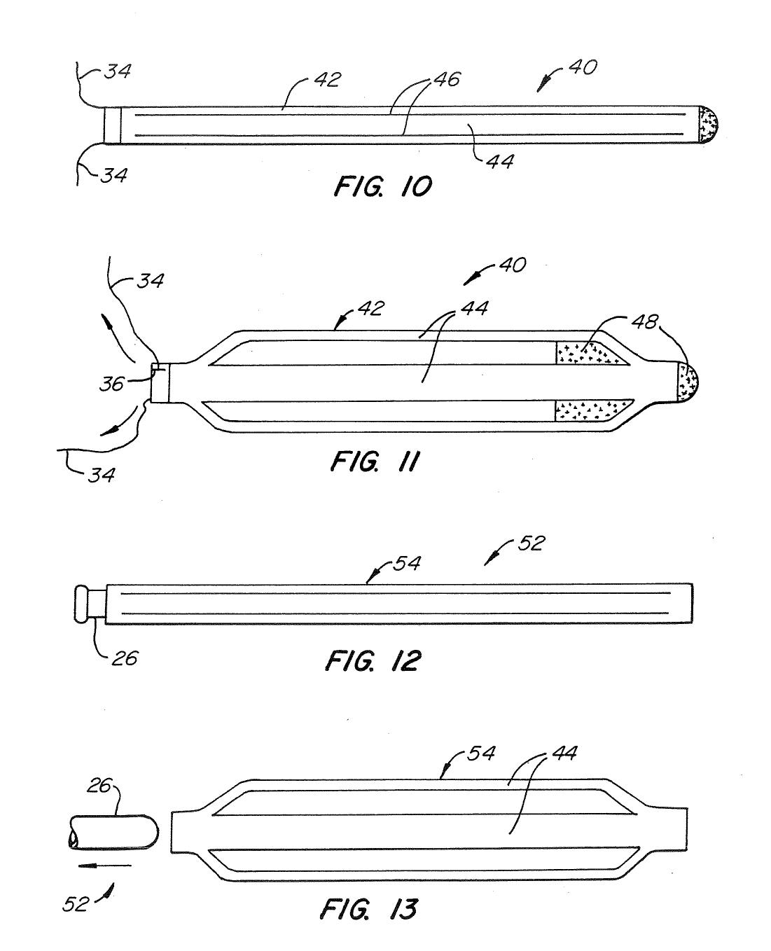

FIGS. 10 and 11 illustrate a fourth embodiment of the invention. Device 40 is similar to device 30 of FIG. 8 in that it naturally assumes a relaxed, radially contracted position as shown in FIG. 10. However, instead of being made from a tubular braided element, the tubular element is a Malecott-type tubular element 42. Tubular element 42 includes four arms 44 separated by slits 46. String-like placement elements 34 are used, as in the embodiment of FIGS. 8 and 9, to place tubular element 42 in a radially expanded position. Device 40 also has a porous filter section 48. Section 48 is preferably made of a radially collapsible and expandable material, such as a sponge-like material or a cotton ball-like material, which permits the relatively unhindered flow of air along the air passageway when tubular element 42 is radially expanded state while filtering the air and/or treating tissue along the air passageway with an agent.

Porous filter sections 28, 48 are shown located at the distal end of the tubular element. However, a filtering device may be located anywhere in the upper airway; for example, the filtering device may be very close to the nostril or anywhere else along the airway. The filter element may be a smaller than those shown or of other shapes, such as spherical, conical, bell-shaped, goblet shaped, etc., as opposed to the shapes of filter sections 28, 48 shown in the figures. The filtering device may also contain a one-way valve in it so that when the person exhales through the upper airway, the valve opens to allow the expelled air to escape easily and not requiring it to pass through the filtering element. These one-way valves are commonly known to those normally skilled in the art such as, but not limited to a duck valve, flapper valve, etc. For example, the narrowed distal end 12 of tubular element 24 of FIGS. 6 and 7 could comprise a one-way valve 12 for this purpose.

FIGS. 12 and 13 show fifth embodiment of the invention in which the device 52 includes a Malecott-type tubular element 54, similar to tubular element 42 in FIGS. 10 and 11, but which assumes a relaxed, expanded condition. Therefore, for insertion and removal, tubular element 54 is placed in the radially contracted condition of FIG. 12 using elongate placement element 26 similar to that of FIGS. 6 and 7. Insertion of placement element 26 through tubular element 54 causes the distal end of the placement element to engage distal end 12 of tubular element 54 thus causing the tubular element to move to its collapsed, radially contracted condition of FIG. 12.

The various upper airway devices discussed above are designed to maintain patency (that is, an open airway), filter air, treat air and deliver agents. While the various embodiment discussed above have been described as typically accomplishing one or two of these functions, devices made according to the invention can be constructed to accomplish one, some or all of these functions.

If designed for systemic use, the tubular element may be configured so that it is placed only within the nose, and does not primarily act as a splint for the airway. An embodiment of the tubular element for systemic delivery of therapy via the nasal mucosa may be a disposable single use item containing a specific amount of drug or other therapy, which may be placed in the nostril for a specific time. Alternatively, the tubular element may be soaked in a solution of a drug or therapy before administration. The tubular element may be used for slow infusion of a therapy for absorption by the nasal mucosa. One aim is to maximize efficacy and cost-effectiveness by developing tubular elements that consistently deliver the right amount of drug to the right part of the nasal cavity. When the tubular element is designed to reside completely within nasal cavity 17, the device may be designed to be easily retrievable, such as through the use of a retrieval string extending from proximal end 10 through nostril 16. The device may be designed to remain within the upper airway permanently or semi-permanently or to be retrievable by medical personnel and also may be designed to be bioabsorable or biodegradable.

The tubular element may be constructed to facilitate electrical stimulation, iontophoresis, delivery of radiowaves and other forms of electromagnetic energy, vibratory and mechanical energy delivery, or radiation. The shape and size of the tubular mesh braid or other porous tubular element may vary to accommodate different anatomy. Additional embodiments may be used with other modalities, i.e., CPAP, to treat sleep apnea. Portions of the porous tubular element may be covered with a membrane to prevent positive airway pressure from negatively affecting the sinuses.

Still further embodiments consist of an expandable endotracheal or nasotracheal tube. A flexible and expandable nasogastric tube also may be created in a similar manner.

Proximal end 10 of the tubular element of may be enlarged or otherwise configured to help prevent the proximal end of the tubular element from entering nasal cavity 17. A retention device (not shown) may comprise a tether, which is attached to proximal end 10 of the tubular element and draped to the sides, below the ears and around the neck. This would prevent the tubular element from being dislodged into the hypophanynx and causing obstructive problems. Another configuration (not shown) may be to extend the tubular mesh braid from the pharynx, through the nostril and nares so that the external end is externalized. The flaring of the mesh braid external to the nares will serve as a tether to keep the device in place. In fact, the mesh braid may be formed so that the external flaring actually folds comfortably over the nose in a low profile manner, essentially creating a nose mask. Still another configuration (not shown) may be to place the external end of the mesh braid into tension. This would collapse the tubular structure into a string like structure. By adding any one of a number of substances to it, the extended or collapsed configuration may be maintained by essentially gluing the filaments together. This would create a mandrel for pushing the mesh braid from the catheter for deployment, for securing it in place during deployment, and for retrieval after use. Other retention devices (not shown) may include a modified V-clip, an adhesive strip, or other means of securing the device.

Other modification and variation can be made to the disclosed embodiments without departing from the subject of the invention as defined in following claims. For example, the tubular element may include a multiple filter sections 28; some or all of the filter sections may be made, for example, as funnel-shaped tubular braided filter sections similar to that shown in FIG. 7. The string-like placement elements 34 may be routed outside the tubular element or a combination of outside and inside the tubular element. Also, elongate placement element 26 could be configured to extend outside of the tubular element to place the tubular element in tension; for example, elongate placement element 26 could be flexible, hollow tube housing the tubular element therein or it could be a solid shaft against which a porous braided mesh-type tubular elements lies against and wraps around.

Any and all patents, patent applications and printed publications referred to above are incorporated by reference.

1. A device for delivering an agent to tissue of a user comprising:
a radially expandable tubular element having proximal and distal ends;
the tubular element sized to pass through a nostril and into the user's upper airway to be positioned at a desired location within the user's upper airway so that when the tubular element is radially expanded, an air passageway is created within the support element between the proximal and distal ends; and
the tubular element comprising a body material and an agent releasable from the body material when the tubular element is radially expanded.

2. The device according to claim 1 wherein the agent is releasable into tissue lining a user's upper airway and in contact with the tubular element.

3. The device according to claim 1 wherein the agent is releasable into air passing through the air passageway for delivery to tissue at one or more locations along a path extending from the upper airway to the user's lungs.

4. The device according to claim 1 wherein the tubular element comprises a radially expandable and contractible tubular element.

5. The device according to claim 4 further comprising means for selectively placing the tubular element in a radially contracted condition and a radially expanded condition.

6. The device according to claim 5 wherein the tubular element assumes a radially expanded condition when in a relaxed state.

7. The device according to claim 6 wherein the selectively placing means comprises a flexible insertion sheath within which the tubular element is housed to place the tubular element in a radially contracted condition.

8. The device according to claim 6 wherein the selectively placing means comprises an elongate placement element engageable with the distal end of the tubular element to place the tubular element in tension and into a radially contracted condition.

9. The device according to claim 5 wherein the tubular element assumes a radially contracted condition when in a relaxed state.

10. The device according to claim 9 wherein the selectively placing means comprises an elongate placement element secured to the distal end of the tubular element and extending at least to the proximal end of the tubular element so that pulling on the placement element relative to the proximal end of the tubular element places the tubular element in compression causing the tubular element to assume a radially expanded condition.

11. The device according to claim 10 wherein the elongate placement element comprises a string-like element and the selectively placing means further comprises a catch element at the proximal end of the tubular element to which the string-like element is securable.

12. The device according to claim 1 wherein the tubular element is a porous mesh tubular element.

13. The device according to claim 1 wherein the body material is a bioabsorable or biodegradable body material.

14. The device according to claim 1 wherein the agent is a drug therapy agent.

15. The device according to claim 1 wherein the agent is a radiation therapy agent.

16. The device according to claim 1 wherein the agent is a gene therapy agent.

17. The device according to claim 1 wherein the agent is a mechanical therapy agent.

18. The device according to claim 1 wherein the agent is an electromagnetic therapy agent.

19. The device according to claim 1 further comprising a balloon expandable within the tubular element.

20. A method for delivering an agent to tissue of a user comprising:
selecting a radially expandable tubular element having proximal and distal ends;
placing the tubular element through a nostril and into the user's upper airway;
positioning the tubular element at a desired location within the user's upper airway;
radially expanding the tubular element thereby creating an air passageway within the tubular element between the proximal and distal ends;
the selecting step further comprising choosing a tubular element comprising a body material and an agent releasable from the body material; and
releasing the agent for delivery to tissue of the user.

21. The method according to claim 20 wherein the releasing step comprises releasing the agent for delivery into tissue lining a user's upper airway at the desired location.

22. The method according to claim 20 wherein the releasing step comprises releasing the agent into air passing through the air passageway for delivery to tissue at one or more locations along a path extending from the upper airway to the user's lungs.

23. The method according to claim 20 wherein the releasing step comprises releasing the agent into tissue contacting the tubular element.

24. The method according to claim 20 wherein the selecting step comprises selecting a tubular element of the type which assumes a radially contracted condition when in a relaxed state.

25. The method according to claim 20 wherein the selecting step comprises selecting a porous tubular element.

26. The method according to claim 20 further comprising:
radially contracting the support element; and
removing the radially contracted support element through the nostril.

27. The method according to claim 20 wherein the choosing step is carried out using a bioabsorable or biodegradable body material for the tubular element.

28. The method according to claim 20 wherein the choosing step is carried out with the agent being a drug therapy agent.

29. The method according to claim 20 wherein the choosing step is carried out with the agent being a radiation therapy agent.

30. The method according to claim 20 wherein the choosing step is carried out with the agent being a gene therapy agent.

31. The method according to claim 20 wherein the choosing step is carried out with the agent being a mechanical therapy agent.

32. The method according to claim 20 wherein the choosing step is carried out with the agent being an electromagnetic therapy agent.

33. The method according to claim 20 wherein the radially expanding step comprises expanding a balloon within the tubular element.

34. The method according to claim 20 further comprising expanding a balloon within the tubular element after the radially expanding step.
